(19) United States
(12) Patent Application Publication
(10) Pub. No.: US 2017/0148214 A1
(43) Pub. Date: May 25, 2017

(54) VIRTUAL REALITY TRAINING

(71) Applicant: IVD MINING, Las Condes, Santiago (CL)

(72) Inventors: Fernando Morera Muniz-Simas, Santiago (CL); Silvia Regina Marega Muniz-Simas, Santiago (CL)

(21) Appl. No.: 14/762,434

(22) PCT Filed: Jul. 17, 2015
(86) PCT No.: PCT/US2015/041013

§ 371 Date: Jul. 21, 2015

(51) Int. Cl. G06T 19/00; G06F 3/14; A61B 5/16; G05B 9/00; G09B 9/00; A61B 5/024; G06F 3/01; G09B 19/24

(52) U.S. Cl. CPC G06T 19/006; G06F 3/012; G06F 3/013; G06F 3/017; G06F 3/015; G06F 3/14; G09B 19/24; G05B 9/00; G09B 9/00; A61B 5/024; A61B 5/165; G06F 2203/011; G06T 13/40

(57) ABSTRACT

A virtual reality training system for industrial labor applications is disclosed. Users wear virtual reality equipment including a head mounted device and enter a virtual worksite replete with VR industrial equipment, VR hazards, and virtual tasks. Through the course of completing the tasks a plurality of sensors monitor the performance of the user or users and identify knowledge gaps and stresses of the user(s). The system generates an evaluation associated with the user(s) and then informs the user where there is room for improvement and informs an administrator of potential liabilities latent within evaluated employees.

### CROSS REFERENCE TO RELATED APPLICATIONS

This application is a 35 U.S.C. 371 national stage application of PCT Application No. PCT/US2015041013, filed Jul. 17, 2015. No amendments have been made to the cited International Application.

### TECHNICAL FIELD

Embodiments of the invention relate to the use of virtual reality to provide training modules. The embodiments more particularly relate to the use of a plurality of sensors to capture actions in an immersive virtual work environment and evaluate the ability of a worker.

### BACKGROUND

Virtual reality simulations are used in a plurality of applications. These simulations vary in quality, immersion, scope, and type of sensors used. Some applications include the use of head mounted devices (HMDs), which track the wearer as he navigates through a mapped out space or a room. Locations within the mapped out space correspond to locations within a virtual world. By pacing through the mapped out room, the wearer is enabled to interact with virtual creations.

### BRIEF DESCRIPTION OF THE DRAWINGS

FIG. 1 is an illustration of a user wearing a head mounted device in a mapped out room, according to various embodiments;

FIG. 2 is an illustration of a head mounted device, according to various embodiments;

FIG. 3 is a block diagram of a virtual reality system, according to various embodiments;

FIG. 4 is an illustration of a user wearing a head mounted device and viewing virtual constructs, according to various embodiments;

FIG. 5 is an illustration of a user wearing a head mounted device and adjusting position in order to observe virtual constructs, according to various embodiments;

FIG. 6 is a flow chart of a virtual reality safety training program, according to various embodiments;

FIG. 7 is an illustration of a virtual worksite, according to various embodiments;

FIG. 8 is an illustration of a first embodiment of a peripheral control;

FIG. 9 is an illustration of a second embodiment of a peripheral control;

FIG. 10 is an illustration of a multi-player function wherein all users are in the same room, according to various embodiments; and

FIG. 11 is an illustration of a multi-player function wherein users are located remotely, according to various embodiments.

### DETAILED DESCRIPTION

Resource extraction worksites are dangerous. Workers use enormous machinery, flammable materials, and powerful electric currents on a regular basis. Such risks pose a significant danger to both human health and property. Accordingly, employing trained and competent workers is of paramount concern to organizations in industrial fields. Training methods involving greatly reduced risk are therefore valuable. Embodiments of the invention thus include virtual reality simulations to evaluate and correct the knowledge gaps of and latent risks to heavy industrial employees. Further, in some cases provide work certifications to passing employees.

Examples of resource extraction fields are mining, oil and gas extraction, and resource refining. However, other fields are suitable for virtual reality training. Examples of such other fields include raw material generation (incl. steel, radioactive material, etc.), manufacturing of large equipment (incl. airliners, trains, ships, large turbines, industrial machines, etc.), and large-scale construction (incl. bridges, elevated roadways, sky-scrapers, power plants, utility plants, etc.).

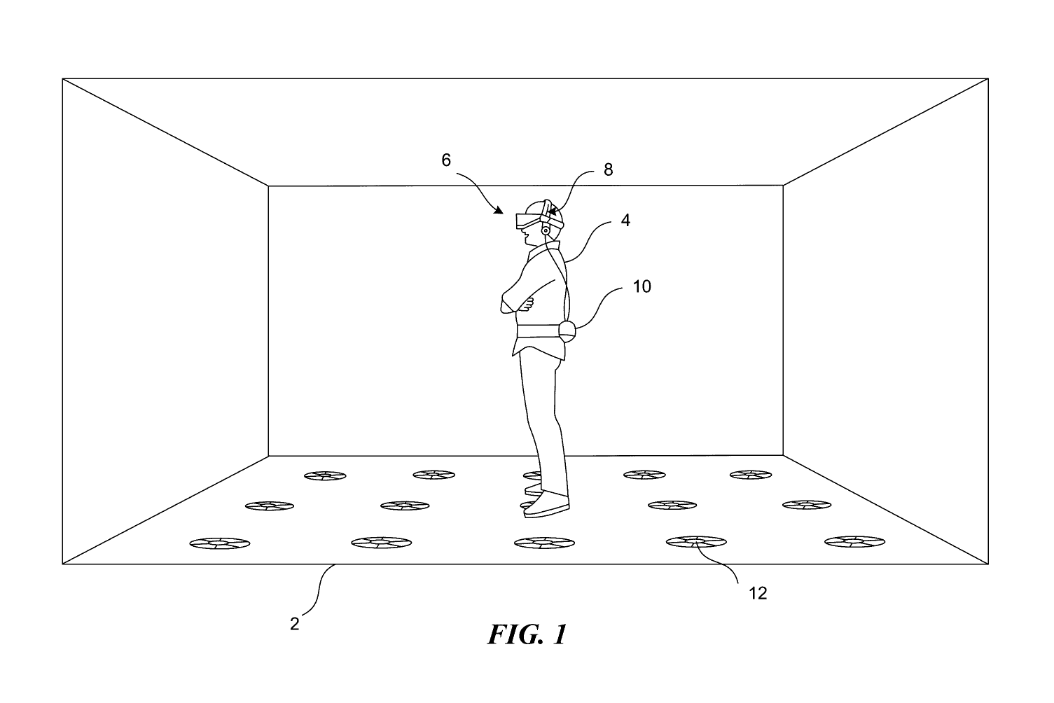

FIG. 1 is an illustration of a user wearing a head mounted device (HMD) in a mapped out room, according to various embodiments. To generate a virtual reality training simulation, an administrator sets up a mapped space 2. Examples of a mapped space 2 include a room or an outdoor area. The mapped space 2 corresponds to a virtual worksite. The virtual worksite is displayed to a user 4 by use of a virtual system 6. The virtual system comprises at least a head mounted device 8 and a processor 10. In various embodiments, the location of the processor 10 varies, though example locations are body mounted, remote, or incorporated inside the HMD 8. In some embodiments, the navigable space in the virtual worksite is the same size as the mapped space 2. In other embodiments, the navigable space in the virtual worksite takes up a different scaled size. Accordingly, in these embodiments, a single step in one direction in the mapped space 2 corresponds to a larger or smaller movement within the virtual worksite.

The navigable space of the virtual worksite refers to everywhere a user can virtually stand in the virtual worksite. In some embodiments, the virtual worksite is massive in size, and although the user 4 is enabled to view virtual vistas within the virtual worksite, the user 4 is not enabled to actually visit all of these virtual locations.

In order to correspond movement in the mapped space 2 to movement in the virtual worksite, the virtual system 6 tracks the movement of the HMD 8. In some embodiments, the HMD 8 uses peripheral capture devices to image a plurality of floor markings 12. The HMD 8 is enabled to determine the location in the mapped space based on positioning relative to the floor markings 12. In some embodiments, the HMD 8 is tracked by exterior cameras mounted on the bounds of the mapped space 2. In some embodiments, the HMD 8 includes a GPS tracker that determines the location of the HMD 8 relative to the mapped space 2. In some embodiments, the user 4 wears foot sensors and the user 4 is tracked according to distance from a static chosen point. Other means of tracking the HMD 8 relative to the mapped space 2 are suitable and known in the art.

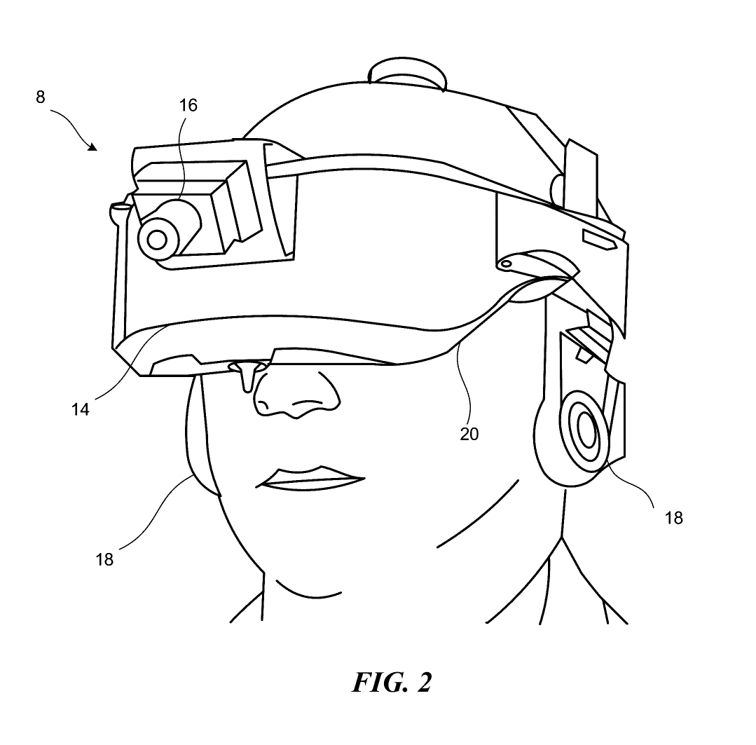

FIG. 2 is an illustration of an HMD 8, according to various embodiments. The HMD 8 includes numerous components. In various embodiments of an HMD 8, the HMD 8 includes some or all of the following: a VR lens 14, a motion capture system 16, speakers 18, and an eye tracking sensor 20.

There are many suitable HMD models available. Examples of suitable HMDs are the zSight, xSight, and piSight head mounted devices as marketed by Sensics, Inc. of Columbia, Md. There are many suitable examples of eye tracking sensors 20 as well. An example of a suitable eye tracking sensor is the ViewPoint Eye Tracker marketed by Arrington Research, Inc. of Scottsdale, Ariz.

There are many suitable motion capture systems 16 available. Examples of acceptable motion tracking systems are those systems manufactured under the brand name InterSense, by Thales Visionix, Inc. of Aurora, Ill. Some motion capture systems 16 are a composite of multiple sensors. Composite systems may use one sensor for hand gesture tracking and one sensor for movement relative to the mapped space 2. Suitable examples of a sensor dedicated to hand gesture tracking includes either the Leap Motion sensor marketed by Leap Motion, Inc. of San Francisco, Calif., and/or the Gloveone marketed by Gloveone of Almeria, Spain. Accordingly, the motion capture systems 16 include any of: cameras, heat sensors, or interactive wearables such as gloves.

These components are incorporated together to provide the virtual system 6 with much data about the user 4 and to enable the user 4 to interact with the virtual worksite. The motion capture system 16 is utilized to both track the motion of the HMD 8, as well as track gestures from the user 4. In various embodiments, the gestures are used to direct virtual constructs in the virtual worksite and/or enable the user 4 to control the user interface of the HMD 8.

The eye tracking sensor 20 is mounted on the inside of the VR lens 14. The eye tracking sensor 20 is used in combination with the motion capture system 16 to determine what virtual constructs the user 4 is looking at in the virtual worksite. Provided location information for the HMD 8, the virtual system 6 is enabled to establish what is in the user's vision. Then, provided with the trajectory of the user's eye, the virtual system 6 is enabled to calculate based on the available data which virtual constructs the user 4 is looking at.

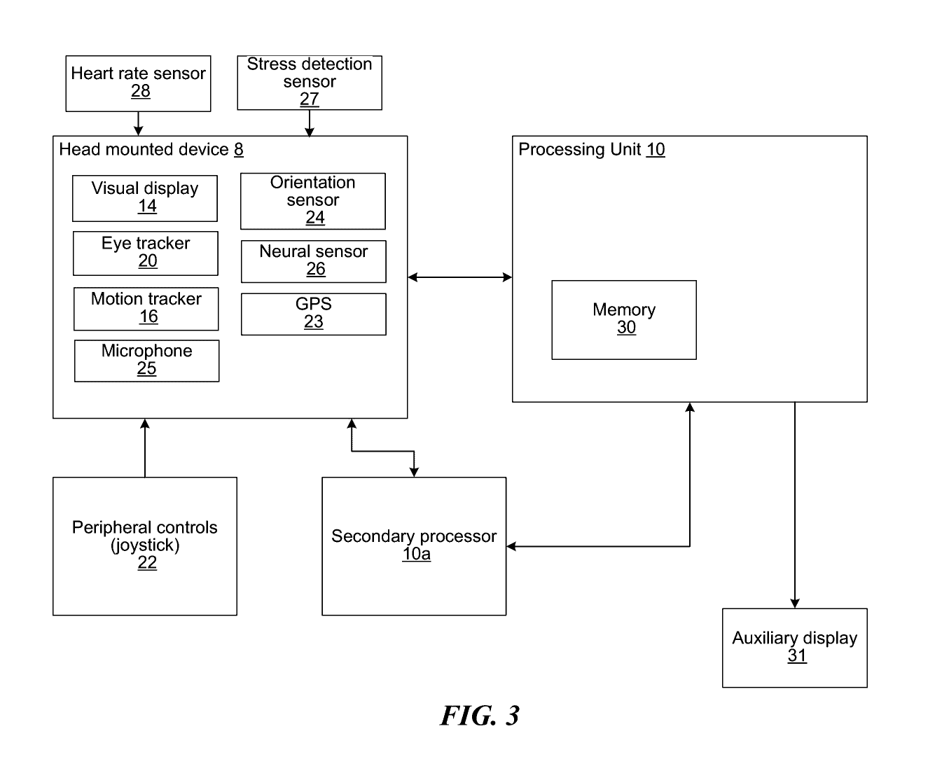

FIG. 3 is a block diagram of a virtual reality system 6, according to various embodiments. In some embodiments, the virtual system 6 includes additional components. As previously stated, the virtual system 6 includes an HMD 8 and a processor 10. In various embodiments, the virtual system 6 additionally includes one or more of a secondary processor 10a, a peripheral control 22, a GPS 23, an orientation sensor 24, a microphone 25, a neural sensor 26, a stress detection sensor 27, a heart rate sensor 28, and/or a memory 30.

The processor 10 and the secondary processor 10a share the load of the computational and analytical requirements of the virtual system 6. Each sends and receives data from the HMD 8. In some embodiments, the processor 10 and the secondary processor 10a are communicatively coupled as well. This communicative coupling is either wired or wireless. The locations of the processor and secondary processor 10a vary. In some embodiments, the secondary processor 10a is body mounted, whereas the processor 10 is housed in a computer in a remote location.

The peripheral control 22 refers to a remote control associated with industrial equipment. In some embodiments, the peripheral control 22 includes a joystick. The orientation sensor 24 determines the gyroscopic orientation of the HMD 8 and enables the HMD 8 to determine the angle the user 4 is looking. The GPS 23 aids in detecting movement of the HMD 8. The orientation sensor 24 is included on a plurality of suitable HMD 8 devices available. The microphone 25 enables users 4 to provide auditory cues when applicable to tasks performed on the virtual worksite. The auditory cues received by the microphone 25 are processed by the virtual system 6 and are a source of simulation data. The motion tracker 16, eye tracker 20, peripheral controls 22, GPS 23, orientation sensor 24, and microphone 25 improve the immersiveness of the virtual worksite and provide contextual data for actions performed by the user 4 within the virtual worksite.

The neural sensor 26 is affixed inside the HMD 8 and monitors brain activity of the user 4. The stress detection sensor 27 is in contact with the user 4 and measures the user's skin conductance to determine stress levels. The heart rate sensor 28 is in contact with the user 4 at any suitable location to determine the user's heart rate. Neural sensors 26, stress detection sensors 27, and heart rate sensors 28 provide data concerning the well-being of the user 4 while interacting with elements of the virtual worksite. Data concerning which elements stress or frighten the user 4 is important towards either correcting these issues or assigning work to the user 4 which is more agreeable. Sensors 22, 23, 24, 25, 26, 27, and 28 enable the virtual system 6 to create a more immersive virtual worksite and provide additional data to analyze and generate evaluations for the user 4.

The memory 30 is associated with the processor 10 and stores data collected by sensors associated with and communicatively coupled to the HMD 8. The memory 30 further stores the virtual worksite program, which the virtual system 6 runs for the user 4. The memory 30 additionally contains a grading rubric of best practices for the user 4. The actions of the user 4 in the virtual worksite are compared to and judged against this rubric.

The auxiliary display 31 is not affixed to the user 4. Rather, the auxiliary display 31 enables an evaluator (not shown) of the user 4 to see the user's experience. The auxiliary display 31 presents the same images of the virtual worksite that are displayed on the VR lens 14 at a given point in time.

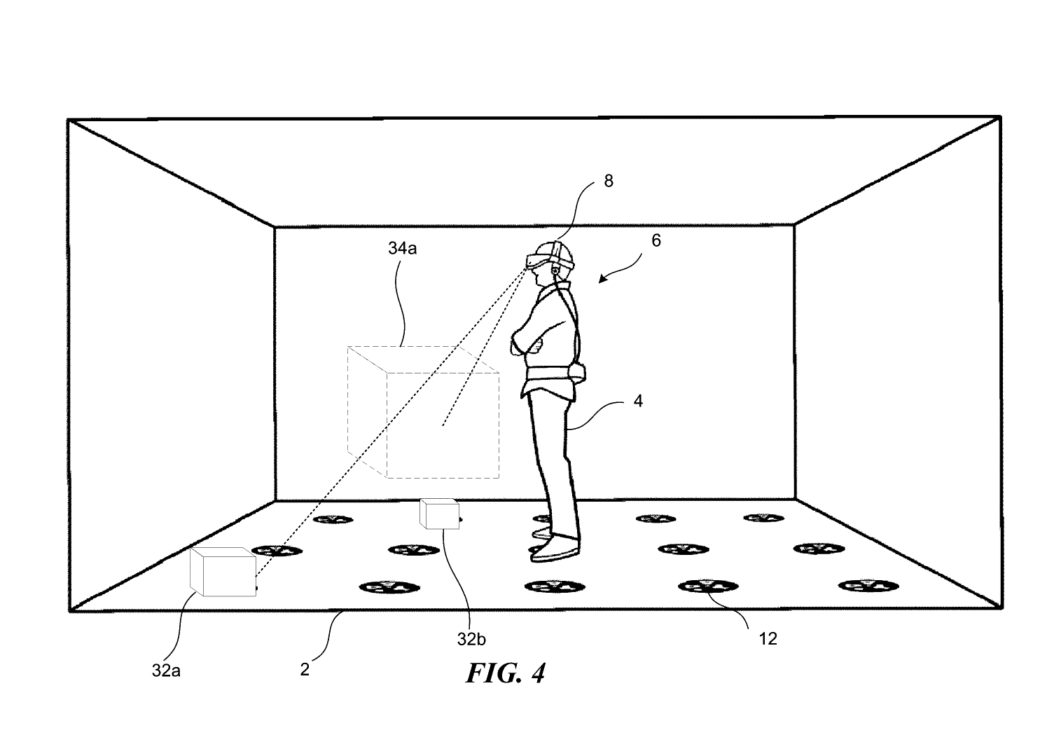

FIG. 4 is an illustration of a user 4 wearing a head mounted device 8 and viewing virtual constructs, according to various embodiments. Virtual constructs take many shapes and roles. A virtual construct is anything displayed to the user through the HMD 8 within the virtual worksite. Some of the virtual constructs are intended to be interacted with. Interaction includes collecting data from sensors associated with and peripheral to the HMD 8 regarding the virtual construct. The interactable virtual constructs are referred to as important safety regions (ISRs) 32 for the purposes of this disclosure. ISRs 32 are zones within the virtual worksite that contain virtual constructs that are important to the simulation the virtual system 6 is carrying out for the user 4.

Other virtual constructs do not directly affect the user's interaction with the virtual worksite. For the purposes of this disclosure, the non-interactable virtual constructs are referred to as obstructions 34. Obstructions 34 serve to block the user's virtual view of important safety regions 32 and to set the scene and provide graphical immersion inside the virtual worksite. In some cases, obstructions additionally prevent the user 4 from progressing forward in the virtual worksite. While the user 4 is able to walk forward in the mapped space 2, the position of the user 4 in the virtual worksite is stalled. In other cases, there are no virtual collisions in order to prevent mapping issues in corresponding a virtual user to the real user 4.

In some cases, merely looking at an important safety region 32 will trigger a response from the virtual system 6, whereas the same behavior with an obstruction 34 does not cause the same effect.

FIG. 4 depicts a user 4 within the mapped space 2 and some virtual constructs. Two ISRs 32a and 32b are located on the floor of the virtual worksite. An obstruction 34a blocks the view of the user from seeing important safety region 32b. In an illustrative example in the virtual worksite, the ISR 32a contains a tool that is out of place, and the important safety region 32b contains an oil spill that is obstructed from view by some machinery 34a. At the position of the HMD 8 as depicted in FIG. 4, the oil spill is not observable.

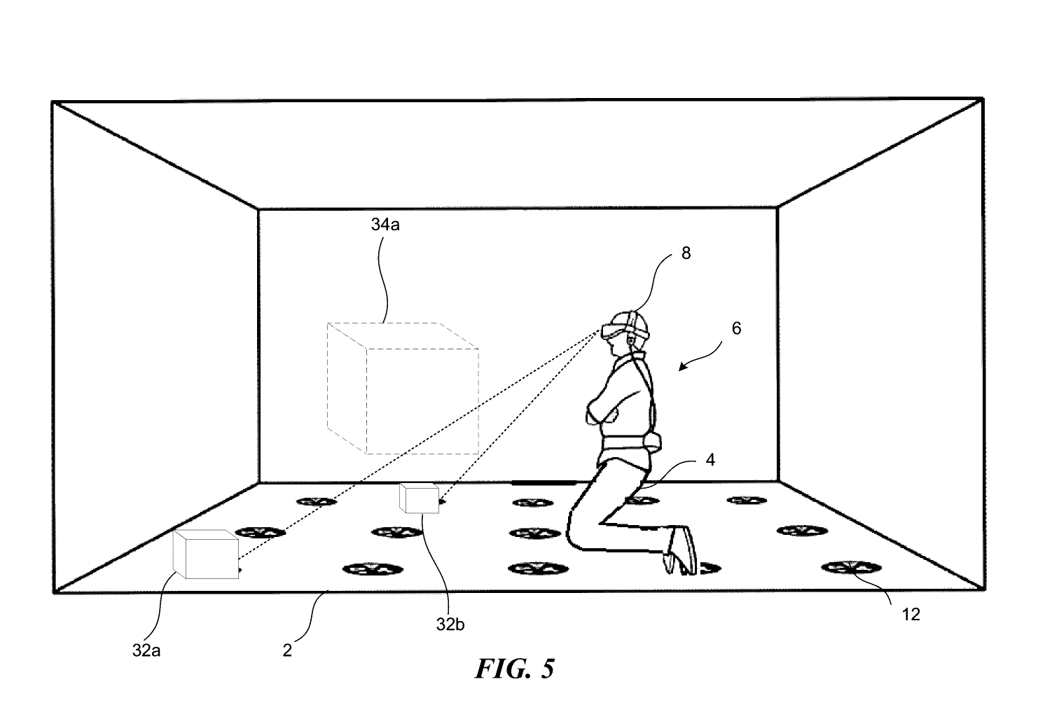

FIG. 5 is an illustration of a user 4 wearing an HMD 8 and adjusting position in order to observe virtual constructs, according to various embodiments. Here, the user 4 is kneeling down and is therefore enabled to see under the obstruction 34a. Due to the position and orientation data collected by the HMD 8 and forwarded to the processor 10 (and 10a), the virtual system 6 displays the ISR 32b. Further, the eye tracking sensor 20 is configured to detect when the user 4 looks at the important safety region 32b.

The virtual system 6 is intended to discover where the user's knowledge gaps are. Returning to the illustrative example wherein the ISR 32a is an out-of-place tool and the ISR 32b is an oil spill, each is directed to a teachable moment. In the case of the out-of-place tool 32a, the sensors on the HMD 8 pick up when the user 4 looks at the tool 32a. There is a trigger in the system noting that the tool 32a was looked at, and behavior of the user 4 is observed concerning the tool 32a. The correct procedure according to a rubric of best practices is for the user 4 to navigate over to the tool 32a and pick up the tool 32a. However, when the user 4 ignores the tool 32a after making eye contact, this demonstrates a knowledge gap in the user's behavior.

In other cases of ISRs 32, such as the oil spill 32b, the rubric of best practices contains multiple components. First, the user 4 must know where to look for the oil spill 32b and then must know to clean up the oil spill 32b. Failure at any level displays a knowledge gap of the user 4. These examples of ISRs 32 serve to illustrate the possibilities of various embodiments of the invention. There are numerous hazards on a worksite, many of which include specific resolution procedures, and all of which are enabled to appear in various embodiments of the virtual worksite.

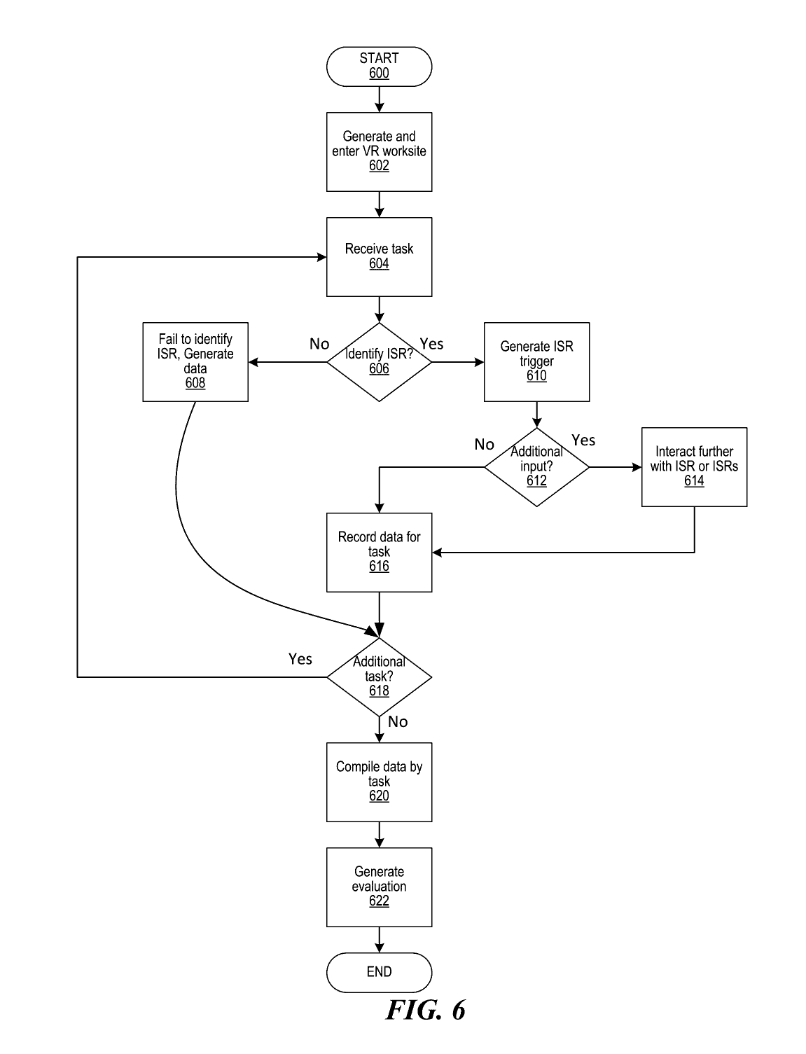

FIG. 6 is a flow chart of a virtual reality safety training program, according to various embodiments. In step 602, the virtual system 6 generates the virtual worksite and the user 4 dons the associated apparatus including the HMD 8. In step 604, the virtual system 6 provides the user 4 with a task. The task is related to the conduct of business within the virtual worksite. The task varies depending on the kind of worksite and the user knowledge elements an administrator chooses to analyze.

In step 606, the virtual system 6 determines whether or not the user 4 identifies a relevant ISR 32. In step 608, when the user 4 does not identify the relevant ISR 32, the virtual system 6 records the data, and the user 4 moves on to the next task if any more exist. When the user 4 does identify the relevant ISR 32, in step 610, the virtual system 6 generates a trigger. The trigger is associated with the relevant ISR 32 and causes additional programming based on the nature of the ISR 32. In step 612, the virtual system 6 determines based on the trigger whether or not the ISR 32 requires additional input. When no, then the task is complete and the virtual system 6 records the task data received by the sensors and moves on to the next task, assuming there are additional tasks.

When yes, then in step 614, the virtual system 6 processes results of the trigger to determine additional actions. Additional actions include receiving input from the user 4 through interface sensors of the virtual system 6 regarding the handling of the ISR 32 or combining input with a first ISR 32 and input from a second, related ISR 32. In step 616, the data collected by the sensors of the virtual system 6 are compiled and organized according to task.

In step 618, the virtual system 6 either assigns an additional task for the user 4 or determines that the simulation is complete. In step 620, when the simulation is complete, all data collected across all tasks is analyzed and compared to the rubric of best practices. In step 622, the virtual system generates an evaluation report for the user 4. The evaluation report includes data concerning the knowledge gaps and strengths of the user. In some embodiments, the report includes data concerning the stresses of the user 4 while carrying out a given task within the simulation.

In some embodiments, particular ISRs or groups of ISRs combined as a task are flagged as critical. Knowledge gaps with respect to these particular ISRs or groups of ISRs impose a harsher evaluation on the user 4. Critical ISRs are those wherein failure to adhere to the best practices rubric corresponds to significant danger of human harm in the physical world.

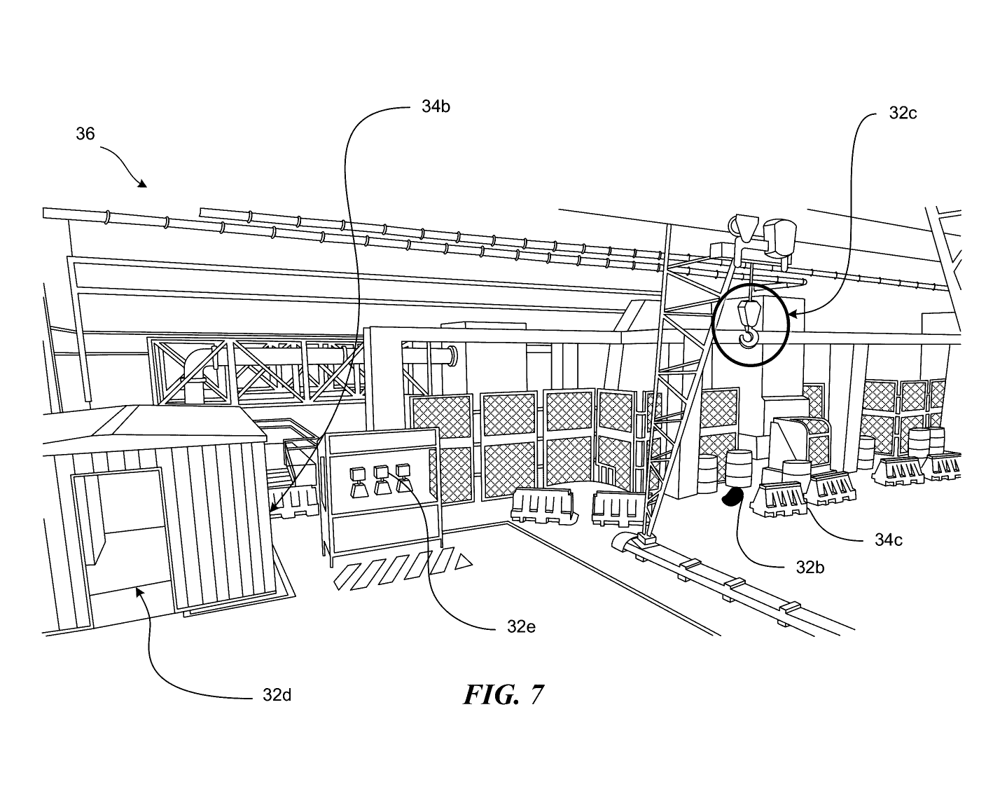

FIG. 7 is an illustration of a virtual worksite 36, according to various embodiments. The virtual worksite 36 corresponds to a mapped space 2, which resides in the physical world. FIG. 7 and the virtual worksite 36 depicted serve as an illustrative example. Other virtual worksites exist and serve other purposes depending on the business employed at the worksite.

In the virtual worksite 36, a user 4 is directed to complete a number of tasks pertaining to a number of ISRs 32 around a number of obstructions 34. In a task to operate a crane 32c safely, the user 4 would make use of a peripheral control 22 to direct the virtual crane 32c according to a best practices rubric. In some embodiments, the best practices rubric for crane operation includes maintaining eye contact with the crane 32c while the crane is in motion. Other practices depend on the nature of the task with the crane 32c.

In another task wherein the user 4 is directed to repair the crane 32c, the user 4 makes use of another ISR 32, the electrical breaker room 32d. In some embodiments, the best practices rubric for crane repair includes electrically locking out the crane 32c before beginning work, to avoid electrocution. In order to complete this task, a user 4 must avoid the walls of the breaker room obstruction 34b. The user 4 is intended to go into the breaker room 32d, correctly identify the breaker for the crane 32c, lock out that circuit, then return to the crane 32c and conduct repairs. Interaction for this task and data collected therein is managed by the eye tracking sensor 20 and hand gestures captured by the motion tracking sensor 16.

Additionally illustrated in FIG. 7 is an oil spill 32b. The oil spill of FIG. 7 is obstructed by a concrete barrier 34c. In some embodiments, tasks regarding ISRs 32 like oil spills 32b are not provided explicit assigned tasks. These tasks are latent, and an administrator of the system attempts to determine if the user 4 is keeping an eye out for latent safety hazards. Other examples of latent hazards include out-of-place tools 32a, puddles near electrical currents, or exposed live wires.

In some embodiments of the virtual worksite 36, the administrator of the simulation wants to include specific safety procedures for a particular site or corporation. Accordingly, the virtual worksite 36 as displayed to a user 4 through the virtual system includes a blockage station 32e. A blockage station 32e is an area where the workers deposit lock keys and a supervisor comes over and blocks the keys in as a secondary measure to avoid the risk of unlocking some equipment that could cause injury.

An example company includes a specific protocol. Because the energies such as mass, pressure, and electricity are so large in mining equipment, blockage keys are used. The key enables a fuse, and without the key, no power is delivered to the equipment. Procedure regarding the blockage station 32e dictates that users 4 lock blockage keys away to demonstrate that a key had not been left behind or plugged into the equipment.

Similarly speaking, in some embodiments, operating a given piece of industrial equipment involves the use of multiple ISRs 32. Such ISRs 32 include checking an ignition to the equipment, checking that all movement areas are clear of objects, and observing for nearby personnel. Missing one of these checks demonstrates a knowledge gap for the user 4.

Additional examples of hazards are typically associated with the task. electrocution, drowning, asphyxiation, burns, and run overs are all associated with the operation of machinery that perform under high pressures, high temperatures, high speeds, or that are substantial in mass and displace vast energies—including mine trucks. Mine trucks have substantial blind spots, and at many angles, the operator cannot see regular trucks on the worksite and simply runs over them. To avoid the run over problem, there are testable procedures.

When performing the task of cutting the energy of large machinery to perform maintenance work, relevant procedures are: affirming that everyone wears the appropriate safety equipment, the electrical room is closed, electrical equipment is isolated, the right equipment is present, and people are trained correctly.

Additional data evaluated concern personal and job-related stresses of the user 4. For example, using a combination of the heart rate sensor 28, the neural sensor 26, and the eye tracker 20, a simulation administrator is enabled to determine stress levels. In some embodiments, the virtual worksite 36 displays a location that is very high up. In related embodiments, the mapped space 2 contains a physical balance beam for the user 4 to walk on. The balance beam is configured at a relatively low height compared to the portrayed location in the virtual worksite 36.

Based upon readings of the biometric sensors associated with the virtual system 6, the simulation administrator can evaluate the user 4 for fear of height, vertigo, and other similar conditions known in the industry. The virtual system 6 provides an opportunity for the administrator to evaluate medical conditions observable by the biometric sensors associated with the virtual system 6 during simulated work. The evaluations of the user 4 by the virtual system 6 provide the administrator data on what elements of work cause stress to a given employee without the employee having to wear monitoring equipment when actually on the job. Rather, the employee is examined during a virtual reality training exercise.

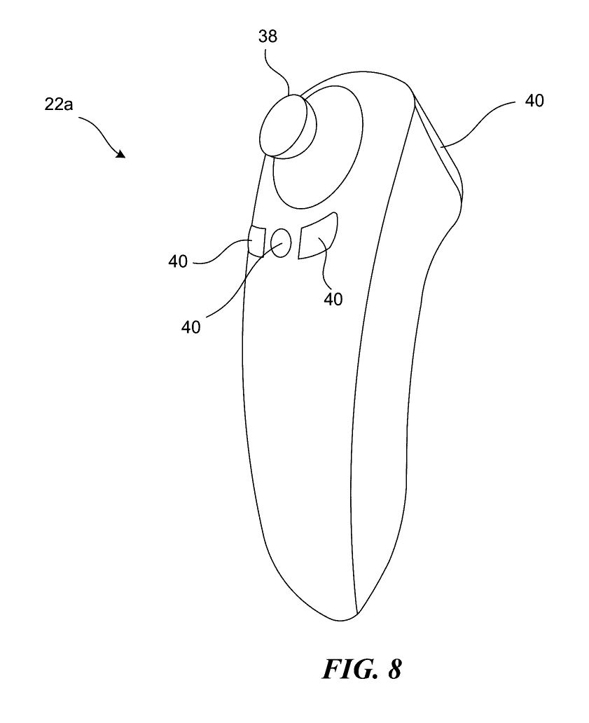

FIG. 8 is an illustration of a first embodiment of a peripheral control 22. The first embodiment of a peripheral control 22a is utilitarian in design. The peripheral control 22a includes a single control stick 38 and several buttons 40. The peripheral control 22a is used to direct simple virtual reality industrial equipment. Virtual reality industrial equipment comprise interactable virtual constructs. In some embodiments, all of, or elements of, virtual reality industrial equipment comprise ISRs 32.

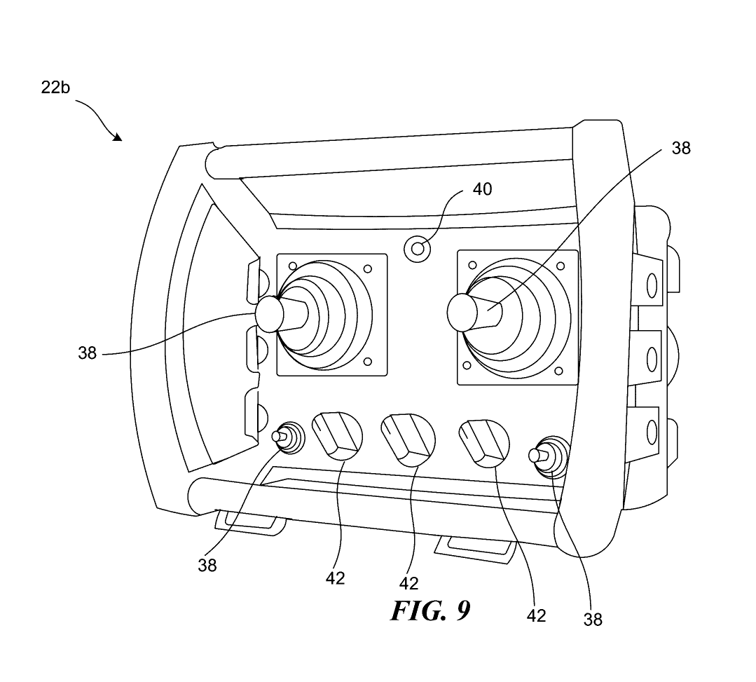

FIG. 9 is an illustration of a second embodiment of a peripheral control 22. The second embodiment of a peripheral control 22b is more complex than the first embodiment of a peripheral control 22a. Peripheral control 22b includes a plurality of control sticks 38, buttons 40 and dials 42. The peripheral control 22b is an illustrative example of a repurposed industrial remote control. Many other configurations of industrial remote controls exist. Industrial remote controls are wireless remotes that connect to industrial equipment (e.g., massive cranes). Industrial remotes are sold and originally configured to connect to wireless receivers on the equipment. For the sake of realism, in some embodiments, the virtual system 6 uses repurposed industrial remote controls. To repurpose an industrial remote control, the transmitter is reconfigured to provide signals generated by actuating or toggling the control sticks 38, buttons 40, and dials 42 to the virtual system 6.

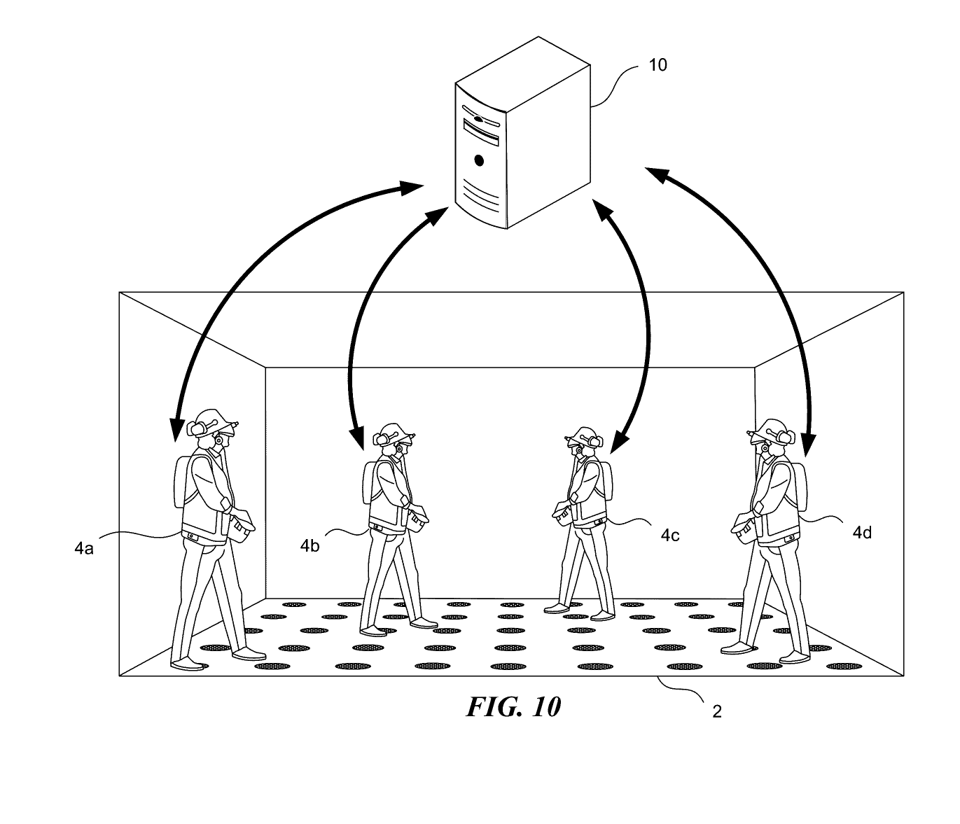

FIG. 10 is an illustration of a multi-user function wherein all users 4 are in the same room, according to various embodiments. In some embodiments, tasks given to a user 4 are better suited given to multiple users 4. FIG. 10 depicts four users 4a, 4b, 4c, and 4d. In some multi-user embodiments, the virtual system 6 includes a processor 10 associated with the HMD 8 of all of the users 4a, 4b, 4c, and 4d. In some embodiments, each user 4a, 4b, 4c, and 4d has a secondary processor 10a mounted to his body. At the conclusion of the simulation, the virtual system 6 generates evaluations for each of the users 4a, 4b, 4c, and 4d individually and/or as a group.

In the virtual worksite, each of the users 4a, 4b, 4c, and 4d has a corresponding avatar representing him. This prevents the users 4a, 4b, 4c, and 4d from running into each other in the physical mapped space 2. The user avatars further enable the users 4a, 4b, 4c, and 4d to more readily carry out the desired simulation. Additionally, in some embodiments, each avatar for each of the users 4a, 4b, 4c, and 4d is considered by the virtual system 6 as an ISR 32, wherein during some tasks, a given user 4 is expected to identify the location of all other users with eye contact detected by the eye tracking sensor 20 before proceeding. In some circumstances, other users are blocked from eye contract by obstructions 34. In some embodiments, the best practices rubric dictates that users 4a, 4b, 4c, and 4d use auditory cues, received by the microphone 25, to verify the location of one another.

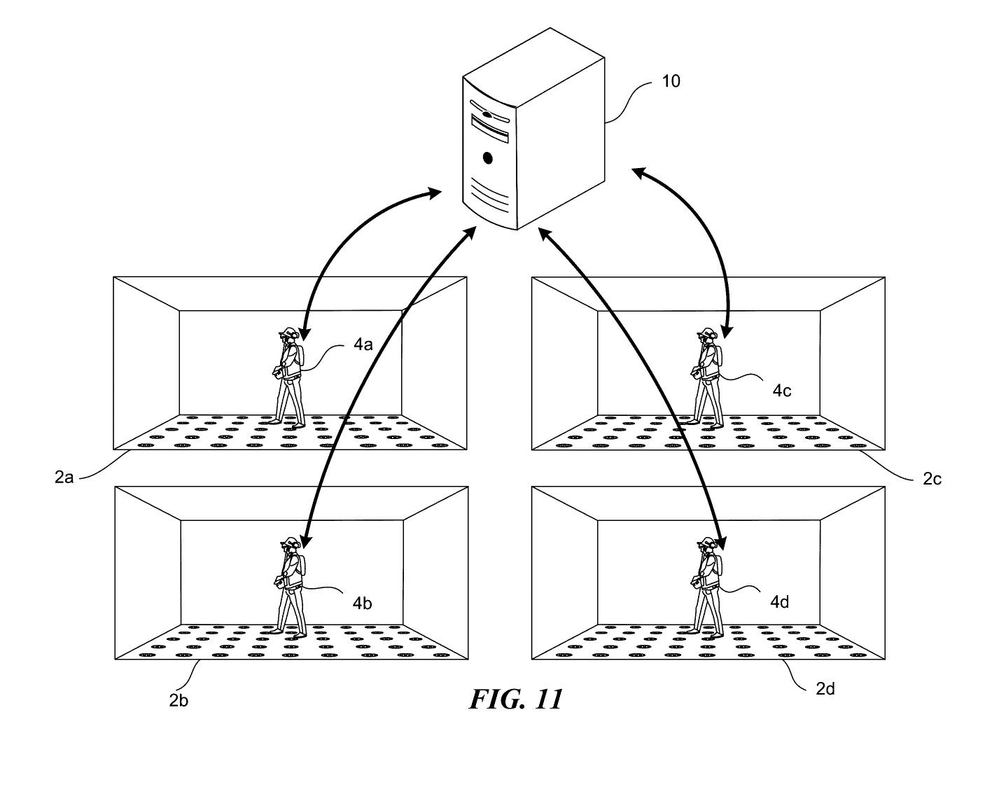

FIG. 11 is an illustration of a multi-user function wherein users 4 are located remotely, according to various embodiments. In some multi-user embodiments, each of the users 4a, 4b, 4c, and 4d is located in individual and corresponding mapped spaces 2a, 2b, 2c, and 2d. In some embodiments, users 4a, 4b, 4c, and 4d enter different virtual worksites 36, wherein the different virtual worksites are within virtual view of one another (e.g., are at differing elevations in the same local virtual area). Accordingly, each of the users 4a, 4b, 4c, and 4d is enabled to see the corresponding avatars of the user users 4, though he cannot occupy the same virtual space of the corresponding users.

1. A method for generating an immersive virtual reality(VR) platform for workers of dangerous mining, oil, and gas worksites to provide training or certification programs replete with a plurality of sensors to detect and correct knowledge gaps and prevent life threatening situations, all confined within the safety of a virtual reality worksite, comprising:
generating a VR resource extraction worksite including virtual dangers and massive virtual industrial machines;
displaying the VR resource extraction worksite to a user with a head mounted device including sensors;
tracking the user with the head mounted device and sensors as the user navigates the VR resource extraction worksite completing tasks and interacting with the virtual dangers and massive virtual industrial machines using a combination of eye contact detection, hand gestures, and heavy machinery remote controls;
identifying incorrect machine procedures and neglected virtual dangers as compared to a rubric of best practices;
collecting biometric data including stress response, heart rate, and fear of the user while the user performs tasks in the VR resource extraction worksite;
generating an evaluation of the user according to the best practices rubric, the evaluation concerning safety procedures, equipment operating procedures, and awareness of latent dangers such as electrocution, burns, downing, impact and crushing hazards; and
presenting the evaluation to the user to improve work performance and safety.

2. A method for virtual reality (VR) training, comprising:
generating, by a processor, a VR heavy industry worksite comprising VR industrial equipment and VR hazards;
displaying the VR heavy industry worksite to a user with a head mounted device including sensors;
tracking the user with the head mounted device as the user navigates the VR heavy industry worksite;
receiving, by the processor, sensor data collected by the sensors, the sensors comprising all of:
an eye tracking sensor;
peripheral controls simulating industrial equipment; and
a motion tracking sensor;
wherein, the sensor data comprises all of:
stress response data associated with the user to the VR resource extraction worksite;
active use procedure data associated with the user interacting with the VR industrial equipment; and
hazard awareness and resolution data associated with the user interacting with the VR hazards;
creating an evaluation associated with the sensor data by the processor according to a best practices rubric;
reporting the evaluation to either a physical display or digital display.

3. The method of claim 2, wherein the VR industrial equipment comprises any of:
virtual equipment associated with oil extraction;
virtual equipment associated with gas extraction;
virtual equipment associated with large scale construction; or
virtual equipment associated with ore or mineral extraction.

4. The method of claim 2, wherein the VR hazards comprise any of:
virtual oil spills;
virtual oil leaks;
virtual misplaced tools;
virtual improperly balanced objects;
virtual lack of proper equipment;
virtual electrical systems;
virtual contact with electrical sources;
virtual contact with high pressures;
virtual contract with high temperatures sources;
virtual work at heights;
virtual contact with mobile equipment; or
virtual contact with radiation.

5. The method of claim 2, wherein the head mounted device is configured to detect vertical motion of the user, and said VR hazards are situated at variable heights within the VR heavy industry worksite, and said best practices rubric includes identifying VR hazards at heights other than eye level.

6. The method of claim 5, wherein VR hazards are concealed behind virtual obstructions, and in order to view VR hazards, the user must circumvent the virtual obstructions.

7. The method of claim 2, wherein the stress response data comprises indicators for vertigo or fear of heights

8. The method of claim 2, wherein the motion tracking sensor is enabled to capture position and gesture data of a hand of the user, wherein the position and gesture data influence virtual conditions of the VR heavy industry worksite.

9. The method of claim 2, wherein the VR hazards are classified into sub categories including:
critical; and
non-critical;
wherein critical VR hazards are those which simulate significant danger to human health.

10. The method of claim 2, further comprising:
providing the user with one or more virtual tasks, the virtual tasks simulating work that takes place in a resource extraction worksite, wherein the evaluation is subdivided into each of the one or more virtual tasks.

11. The method of claim 2, wherein the user is a first user, and further comprising:
displaying a plurality of avatars of other users within the VR heavy industry worksite, the plurality of other users operative in the VR heavy industry worksite with the first user and the data collected associated with the first user further augmented by interaction with plurality of avatars of other users.

12. A method for identifying knowledge gaps associated with a user using virtual reality(VR), comprising:
generating, by a processor, a virtual reality resource extraction worksite comprising at least one important safety region, the at least one important safety region is a defined virtual location within the VR resource extraction worksite that is visually distinct to a user;
obtaining, by the processor, from a location aware head mounted device, position data associated with the location aware head mounted device, said position data comprising a location on a three dimensional coordinate plane and an orientation, said position data further corresponding to a location in the VR resource extraction worksite;
displaying the VR resource extraction worksite to the user with the location aware head mounted device according to the position data;
detecting, by an eye tracking sensor, eye contact data associated with the user and the VR resource extraction worksite, the eye tracking sensor affixed to the location aware head mounted device; and
evaluating the user with respect to the at least one important safety region, wherein said evaluating comprises:
detecting by the eye tracking sensor that the user makes eye contact with the at least one important safety region; and
receiving input from the user associated with a virtual condition of the at least one important safety region.

13. The method of claim 12, wherein the VR resource extraction worksite further comprises:
virtual obstructions, the virtual obstructions preventing line of sight between the user and the at least one important safety region, wherein the user is enabled to generate eye contact with the at least one important safety region only when the location aware head mounted device has predefined acceptable position data.

14. The method of claim 12, wherein input from the user identifies the virtual condition as either:
safe; or
requires action; and
further comprising:
when the virtual condition is requires action, receiving input from the user directed towards the virtual condition.

15. The method of claim 12, wherein input from the user is any of:
auditory;
received through a peripheral device;
user hand gestures received by a motion sensor affixed to the location aware head mounted device; and
user selection through eye movement captured by the eye tracking sensor.

16. The method of claim 12, wherein the at least one important safety region comprises a virtual depiction of equipment, and the receiving input from the user associated with a virtual condition comprises the user virtually collecting the equipment.

17. The method of claim 12, further comprising:
classifying the at least one important safety region as critical or non-critical, wherein a critical important safety region simulates a real world condition that significantly endangers human safety.

18. The method of claim 12, wherein the at least one important safety region comprises at least two important safety regions, and further comprising:
providing the user with one or more virtual tasks, the virtual tasks simulating work that takes place in a resource extraction worksite, the virtual tasks including evaluation with respect to two or more important safety regions; and
generating a report of the user, the report associated with performance of the user on the one or more virtual tasks, wherein the report is based on the combination of said evaluation step with respect to two or more important safety regions.

19. The method of claim 12, wherein the user is a first user, and further comprising:
displaying a plurality of avatars of other users within the VR resource extraction worksite, the plurality of other users operative in the VR resource extraction worksite with the first user and wherein the plurality of avatars of other users each comprise an important safety region.

20. A virtual reality training apparatus, comprising:
a head mounted device including:
a motion tracker;
an eye tracker;
an immersive graphic display;
a processor communicatively coupled to the head mounted device;
peripheral controls simulating industrial equipment, the peripheral controls communicatively coupled to the processor; and
a memory communicatively coupled to the processor, the memory containing a best practices rubric and instructions, the instructions configured to cause the processor to generate a VR resource extraction worksite comprising VR industrial equipment and VR hazards, the immersive graphic display to display the VR resource extraction worksite to a user, and to receive data from the motion tracker, the eye tracker, and the peripheral controls simulating industrial equipment, wherein the data comprises all of:
stress response data associated with the user to the VR resource extraction worksite;
active use procedure data associated with the user interacting with the VR industrial equipment; and
hazard awareness and resolution data associated with the user interacting with the VR hazards;
and further causing the processor to create an evaluation associated with the data compared to the best practices rubric, then report the evaluation to either a physical display or digital display.

21. The apparatus of claim 20, wherein the peripheral controls simulating industrial equipment comprises repurposed remote controls for real industrial equipment.

22. The apparatus of claim 20, wherein the processor is body mounted on the user.

23. The apparatus of claim 20, wherein the processor communicates to the head mounted device wirelessly.
